(19) United States
(12) Patent Application Publication
(10) Pub. No.: US 2005/0148829 A1
(43) Pub. Date: Jul. 7, 2005

(54) Facility for importing a machine-readable data model, particularly medical guidelines, into a workflow management system

(76) Inventors: Klaus Abraham-Fuchs, Erlangen (DE); Eva Rumpel, Erlangen (DE); Sven Tiffe, Wien (AT)

Correspondence Address: Attention: Alexander Burke, Esq.;SIEMENS CORPORATION, Intellectual Property Department, 170 Wood Avenue South, Iselin, NJ, 08830, US

(21) Appl. No.: 10/897,104

(22) Filed: Jul. 23, 2004

(30) Foreign Application Priority Data
Jul. 24, 2003 (DE) 10333797.0

(51) Int. Cl. G06F 7/00; G06F 17/30; G06F 19/00; G01N 33/48

(52) U.S. Cl. 600/300; 702/19

(57) ABSTRACT

A facility is for adapting a machine-readable data model, particularly medical guidelines, for import into a clinical workflow management system. The facility includes a mapping system including a work step database, with a hierarchic breakdown of work steps. It further includes at least one of a mapping module which maps work steps in the data model which has been read in, which belong to a different hierarchic level than the level used in the system, by polling the work step database for work steps on the hierarchic level of the system, and/or an association system including a resource database, which contains an association between generic resource titles in the data model and actual resources in the institution. Finally, the facility includes an association module which translates generic resource titles in the data model which has been read in into actual resources in the institution by polling the resource database. As such, it allows automated, computer-aided import of medical guidelines, in particular, into clinical workflow management systems with reduced time and work involvement.

The present application hereby claims priority under 35 U.S.C. §119 on German patent application number DE 103 33 797.0 filed Jul. 24, 2003, the entire contents of which are hereby incorporated herein by reference.

### FIELD OF THE INVENTION

The present invention generally relates to a facility for adapting a machine-readable data model, particularly medical guidelines, for import into a clinical workflow management system in an institution.

### BACKGROUND OF THE INVENTION

In recent years, “medical guidelines” are becoming increasingly significant in the medical services sector. These guidelines are recommendations for action and decisions to the medical personnel, particularly the doctor, which have been drawn up in a broad consensus by superordinate and generally recognized committees in the medical profession.

In parallel with this, medical institutions such as clinics and doctor's practices are making ever greater use of clinical information systems, which also permit automated support for the clinical workflow using “workflow management systems”. In this context, clinical flows are mapped into machine-readable data models, and the entire workflow is supported, monitored and documented by a computer network on the basis of these data models. One example of such a workflow management system is the product SOARIAN® sold by Siemens AG Medical Solutions.

In this context, the term workflow, which is usual in this area, should be understood to mean the flow of work, i.e. a sequential, parallel or conditional sequence of work steps, known as “action steps”, which should be observed by the clinical personnel when a particular event occurs or when particular circumstances arise. In a workflow management system, the workflows are broken up into individual work steps, responsible persons and necessary resources are allocated and requisite data are automatically forwarded and provided. At decision nodes in the workflow, knowledge-based expert systems are used to provide electronic decision support.

In this connection, what are known as resources are equipment, for example an ECG unit or a CT unit, people, for example specialist or care personnel, or infrastructure, such as the intensive care unit, which are required for performing a work step. In the machine-readable data model of such a workflow management system, each work step also has associated output data and input data. Input data are the initial data which are required for performing a work step. Output data are obtained as the result of a work step which has been performed in full.

The increasing significance of medical guidelines makes it necessary for them also to be included in electronically implemented workflow management systems in the individual institutions. In this context, the medical guidelines have in some cases already been conditioned as a machine-readable data model, i.e. the workflow prescribed for performing the guideline is available in the form of input data, work steps, decision rules, requisite resources and output data in the form of an electronically readable flowchart, for example in the form of a Petri network. However, data models of medical guidelines are based on terms and formats from superordinate entities, e.g. from specialist medical companies, which have created them in a generalized, generic form.

In this case, it is not possible to take account of the resources available in a particular medical institution, established workflows etc. The format, the level of detail specified and possibly the terminology of such a machine-readable data model of medical guidelines have therefore needed to be aligned in complex fashion to date for import into a clinical workflow management system in an institution.

In principle, mapping the real clinical workflow in a given institution with the resources available in that institution, for example diagnostic equipment, laboratory, personnel and generally accepted organization rules, onto a data model which is able to process an available workflow engine, such as SOARIAN®, is today still a largely time-consuming and work-intensive consulting service which can be automated only in fractions. Specifically in the case of medical guidelines, it has to date been necessary to observe and adapt the terminologies and formalisms used for aligning database queries, for aligning decision criteria and for aligning the decision-supporting elements manually.

A comparable set of problems arises when a workflow section is optimized in one institution, for example in a clinic, and then needs to be transferred to another institution.

### SUMMARY OF THE INVENTION

An object of an embodiment of the present invention is to specify a facility which is able to adapt a machine-readable data model of medical guidelines or a comparable medical workflow for import into a clinical workflow management system in an institution automatically. An embodiment of the present invention is intended to reduce the time and work involvement for electronically implementing medical guidelines, in particular, in existing workflow management systems in different institutions.

An object may be achieved by way of the facility according to an embodiment of the present application. Advantageous refinements of the facility can be taken from the description below and also from the exemplary embodiments.

An embodiment of the present invention includes at least an interface for reading in the machine-readable data model, a mapping system including a work step database, which contains a hierarchic breakdown of work steps, and a mapping module which maps work steps in the data model which has been read in, which belong to a different hierarchic level than the level used in the clinical workflow management system, by polling the work step database for work steps on the hierarchic level of the clinical workflow management system, and/or an association system comprising a resource database, which contains an association between generic resource titles in the data model and actual resources in the institution, and an association module which translates generic resource titles in the data model which has been read in into actual resources in the institution by polling the resource database, and a provision module for providing a machine-readable data model which has been aligned after handling by the mapping and/or the association module and is suitable for import into the existing workflow management system. In this context, the mapping system and the association system may be provided as alternatives or in combination.

The text below describes the present facility specifically using the example of the import of medical guidelines as a data model. However, the comments naturally also apply to the import of comparable data models into a clinical workflow management system.

### BRIEF DESCRIPTION OF THE DRAWINGS

Further properties, features, advantages and applications of the underlying invention can be found in the subordinate dependent patent claims and also in the description below of two exemplary embodiments of the invention, which are depicted in the following drawings, in which:

FIG. 1 schematically shows the basic design of a workflow management system and of the present facility.

### DETAILED DESCRIPTION OF THE PREFERRED EMBODIMENTS OF THE INVENTION

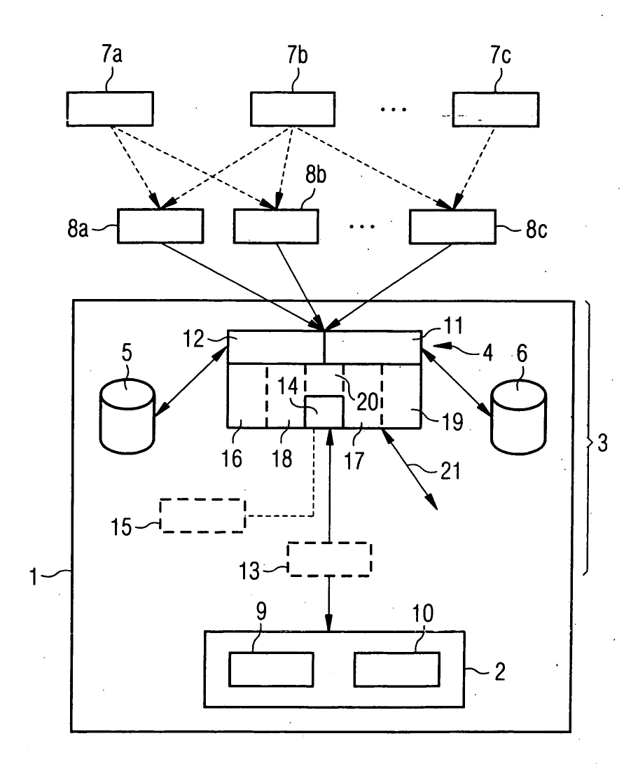

FIG. 1 schematically shows the basic design of a workflow management system and of the present facility. In this case, the workflow management system 2 is implemented together with the present facility 3 in a clinical information system 1, which normally has a computer network. In this arrangement, the present facility 3 includes the modules which have been combined into a unit 4 in this example, the mapping module 11, the association module 12 and the provision module 14, and also the resource database 5 and the work step database 6.

When implementing medical guidelines, from which the present exemplary embodiment illustrates the diabetes guidelines 7a, the stroke guidelines 7b and the X-ray guidelines 7c merely by way of example, these guidelines 7a to 7c may exist in different guideline formats 8a to 8c. The machine-readable data models of these guideline formats 8a to 8c are read in by the present facility 3, as clarified by the arrows in FIG. 1.

Following analysis of the terms, work steps and resources used therein, the resource database 5 and also the work step database 6 are resorted to in order to produce a translation into a data model aligned with the circumstances in an embodiment of the present institution and to implement said translation in the workflow management system 2. This contains the workflow 9 and also a decision support element 10 for executing the guidelines. In this context, implementation in the workflow management system 2 can be effected automatically using the optional incorporation module 13 (which is therefore shown only in dashes in the figure).

The text below provides a more detailed description of further optionally usable modules, a thesaurus module 16, a checking module 17, a scheduling module 18, an interaction module 19 and also a plausibility module 20, which are likewise shown merely in dashes in the figure.

The present facility 3 takes account of the fact that the description of the work steps in the two data models, that of the medical guidelines and that of the actual workflow management system 2 in the present institution, may be available in different degrees of granularity. Thus, by way of example, one model may use a generic term for the work step which, in the other model, corresponds to a chain of two or more work step sections.

By way of example, the guideline at a decision node might demand a blood value, for example the cholesterol level or hematocrit. This is equivalent to the following work steps in the clinical data model: take blood from patient X+request measurement of the blood value Y in the central laboratory+send the blood sample+input the measurement result into the EPR (electronic patient record)+assess the measurement result. Alternatively, this workflow might also be preceded by the work step “poll the EPR for the presence of a current blood value Y”, with the first workflow mentioned being initiated only if the EPR does not contain the data. This example shows the different hierarchic levels to which the specified work steps may belong.

The necessary mapping of the work steps on different hierarchic levels of the description is achieved in the case of the present facility 3 through the provision of the work step database 6, which contains the hierarchic breakdown of the generic terms and associated subterms in a workflow. It also contains the mapping module 11, which compares the work steps in the workflow which is to be imported with the work step database 6 in order to use the associated work steps in the aligned data model's granularity implemented for the institution.

In the same way, a situation may arise in which a medical guideline describes a workflow with a sequence of work step sections which are not provided in this granularity in the institution's workflow. Thus, by way of example, a situation may arise in which the service connected to the work step, for example a magnetic resonance image, cannot be provided in this institution. Instead, the patient has to be transferred to a different institution.

For this case, the hierarchic database 6 may also contain work steps which are not performed in the institution, so that such work steps listed in the guideline can automatically be found and associated with the higher hierarchic level. In this context, the work step database 6 is preferably designed such that each work step it contains has an associated item of information regarding whether this work step is actively implemented in the institution's workflow. This may be done, by way of example, by setting a 0/1 flag for the respective work step in the database 6.

A fundamental part of the implementation of a machine-readable data model for describing a workflow is the allocation and provision of resources which are required for a work step. In this context, the guidelines normally contain the role distribution of responsibility for individual actions in the workflow only implicitly and in a generic manner (“the doctor”, “the laboratory”). In addition, generic titles used in the guideline for resources, such as radiology or ECG monitoring, need to be mapped onto actual titles in the institution, for example the name of the specialists, the unit title, the available infrastructure for ECG monitoring etc.

In particular, each work step needs to have an associated responsible person. For this task, the present facility contains a resource database 5 in which generic role terms or resource titles have associated institution-specific resource titles, for example the names of real departments or functions in the institution, the names of associated specialists or of available medical equipment.

The association module 12 translates the resource titles for the guidelines in the data model which has been read in into the actual resource titles in the present institution by polling the resource database 5. By way of example, the generic term radiology may be allocated the names of the radiology specialists, the term cardiac catheter laboratory may be allocated the unit title and the responsible cardiologist, the term ECG monitoring may be allocated the list of available facilities, such as telemonitoring, central patient monitoring etc.

Finally, the present facility 3 also includes the provision module 14 for providing the machine-readable data model aligned after the mapping and association modules have been encountered. This provision module 14 stores the aligned data model on a suitable data storage medium for the subsequent check, further handling and/or implementation in the clinical workflow management system 2. This implementation is preferably effected using a further module 13 for incorporating the aligned data model into the workflow management system 2 in the institution, so that this incorporation can likewise be effected automatically.

Preferably, the provision module 14 uses a graphical user interface to show the aligned data model to an operator, who can check correct alignment prior to final implementation in the institute's own workflow management system 2. In this regard, FIG. 1 shows an optional monitor 15, connected to the provision module 14, for displaying and comparing the aligned data model with the data model which has been read in. In this case, the display is preferably in a form such that the generic workflow of the initial guideline and the workflow proposed for import can be compared, preferably by simultaneously displaying mutually corresponding components of the two workflows on the monitor 15.

In the aligned data model or workflow proposed for implementation, it is then possible to highlight, for example by way of color marking, which of the subprocesses were able to be translated clearly, in which of the subprocesses a lack of clarity or ambiguities arise and which of the subprocesses were not able to be mapped onto the present institution's data model, for example because they could not be found in the appropriate databases. In addition, the provision module 14 preferably comprises an interface to a user interface in a workflow generation application, so that the operator can correct the proposed workflow and can fill any gaps in the process if appropriate.

The present facility 3 preferably also has a thesaurus module 16 which translates terms used in the medical guideline into institution-specific terms having the same meaning. As a result, any terms used in the medical guideline's generic data model are translated into the sphere of terms for the institution-specific workflow engine. If necessary, the translation is carried out as the first step in adapting the guideline's machine-readable data model. In this context, the thesaurus module 16 preferably includes titles for all of the components in the workflow, i.e. titles for work steps, input and output data and also resources.

In one development of the present facility, an interaction module 19 is also provided which, in the event of a sought work step not being found in the thesaurus 16 and/or in the work step database 6, asks the operator whether this term which is being sought needs to be transferred to the thesaurus 16 or to the work step database 6. Thus, the databases in question can be extended at any time. To this end, the facility includes an appropriate user interface 21 for inputting the new association.

Instead of a direct comparison or in addition to a direct comparison for mapping similar work steps with different titles onto one another, the thesaurus module 16 may also identify a suitable work step or a sequence of work steps using input and output data associated with the work steps in a database. Thus, by way of example, instead of identifying the work steps required for analyzing an X-ray thorax image in an institution, the terms appearing in the guideline for input and output data, e.g. X-ray thorax image as input and conspicuous pulmonary shadow as output, may be used for the search in the database. All of the work steps in a sequential chain between this input and the output can then be implemented as the analysis workflow required for performing this part of the guideline.

In another refinement of the present facility 3, a checking module 17 is provided which checks the guideline for resources which are not available in the institution and proposes alternative available resources and/or a workflow for outsourcing the associated work steps. This assists the operator in defining substitute processes, for example if the guideline for diagnosis demands a resource such as a PET instrument which is not available in the institution. The facility analyzes resource gaps and in so doing automatically proposes alternatives, such as the use of alternative diagnostic methods or a workflow for outsourcing this examination.

In another refinement of the present facility 3, the checking module 17 may provide automated assistance for implementing a defined interface in cases in which subprocesses in the clinical flow do not comply with the guideline. Particularly in relatively small medical institutions, a situation may frequently arise in which not all of the work steps provided in the guideline can be performed in the institution, since necessary resources, for example a cardiology specialist, an MR instrument, etc., are missing and hence the necessary work steps in the institution's data model are not available or cannot be activated.

In this case, the checking module 17 identifies the input and output data associated with the missing work steps and proposes a substitute process, for example an outsource process, which can be used to perform the necessary measures in a different medical institution, for example a specialist practice or a special clinic. To this end, preferably a user interface 21 with a suitable user environment for data input is additionally provided which the operator can use to input any necessary information. Such information may be, by way of example, suitable specialist practices, the responsibility for interfaces between the institutions etc.

A special case of such a substitute process is the frequent case of guidelines in which, although the patient visits his general practitioner as the first port of call, the general practitioner does not have the necessary resources or the necessary specialist knowledge to act in accordance with the applicable guideline, for example for suspicion of stroke. When implementing the guideline, the general practitioner will thus actually include a defined substitute process in his workflow at a very early stage, i.e. upon diagnosis that this guideline is applicable for the patient in question, and will refer the patient to the nearest specialist neurological clinic. In this context, this guideline is then also correct in the general practitioner's workflow system and is implemented in compliance with guidelines.

In another refinement of the present facility 3, a scheduling module 18 is provided which ensures that resources listed in the guideline are available promptly. Thus, by way of example, a guideline may demand that a resource, for example the cardiac catheter laboratory or an MR instrument, be available within a maximum period of time, usually a few hours. When implementing the guideline, it is thus necessary not only to check the presence of the resource, but also to implement processes which guarantee that the resource is available promptly. The scheduling module 18 performs this task (emergency scheduling).

The present facility 3 preferably also includes a plausibility module 20 which automatically compares input and output data in the work steps and also the order of the work steps in the data model which has been read in and the data model which has been aligned. This allows disparities to be identified and to be notified or displayed on a screen to the appropriate operator during or after the performance of the alignment process.

In one refinement of the present facility 3, the checking module 17 is designed such that it constantly or repeatedly checks the availability of resources and, in the event of resources becoming permanently or temporarily unavailable, for example as a result of employees being sick, equipment failing or departments closing, automatically informs the responsible persons about portions of the already implemented guidelines which are affected thereby. In addition, this module 17 may also prompt possibly temporary alignment of the workflow by proposing substitute processes. If the lack of availability is temporary, then the original versions of the guidelines can be automatically reactivated after the resource becomes available again. This refinement of the present facility 3 demands a constant connection to the workflow management system 2 in order to be able to perform the last-mentioned tasks.

The present facility is also suitable for checking the implementability and applicability of medical guidelines in the respective workflow management system in an institution, in advantageous refinements even when the guidelines are not in a foreign format. In particular, it is also possible for authors of the workflow system in the institution to check whether the guidelines which they have designed or formalized can be applied in the system.

The present facility, which is preferably implemented on a computer system, can be used to afford computer-aided import of medical guidelines into clinical workflow management systems. The facility includes modules for analyzing, translating and aligning foreign formats while automatically taking into account available resources. It permits automatic alignment of database queries and decision rules with terminology systems and rule syntax which are used. In addition, advantageous refinements of the facility make it possible to identify lack of resource availability automatically and to locate affected guideline portions. The automated alignment of machine-readable data models of medical guidelines which the present facility permits allow such guidelines to be implemented in actual workflow management systems in different institutions without any great work and time involvement.

Any of the aforementioned methods may be embodied in the form of a program. The program may be stored on a computer readable media and is adapted to perform any one of the aforementioned methods when run on a computer. Thus, the storage medium or computer readable medium, is adapted to store information and is adapted to interact with a data processing facility or computer to perform the method of any of the above mentioned embodiments.

The storage medium may be a built-in medium installed inside a computer main body or removable medium arranged so that it can be separated from the computer main body. Examples of the built-in medium include, but are not limited to, rewriteable involatile memories, such as ROMs and flash memories, and hard disks. Examples of the removable medium include, but are not limited to, optical storage media such as CD-ROMs and DVDs; magneto-optical storage media, such as MOs; magnetism storage media, such as floppy disks (trademark), cassette tapes, and removable hard disks; media with a built-in rewriteable involatile memory, such as memory cards; and media with a built-in ROM, such as ROM cassettes.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A facility for adapting a machine-readable data model for import into a clinical workflow management system in an institution, comprising:
an interface for reading in the data model; and at least one of,
a mapping system including a work step database containing a hierarchic breakdown of work steps, and a mapping module for mapping work steps in the read in data model, which belong to a different hierarchic level than the level used in the workflow management system, by polling the work step database for work steps on the hierarchic level of the workflow management system,
an association system comprising a resource database containing an association between generic resource titles in the data model and actual resources in the institution, and an association module for translating generic resource titles in the read in data model into actual resources in the institution by polling the resource database; and
a provision module for providing a machine-readable data model, aligned after handling by at least one of the mapping system and the association system.

2. The facility as claimed in claim 1, further comprising a module for incorporating the aligned data model into the workflow management system in the institution.

3. The facility as claimed in claim 1, further comprising a thesaurus module for translating terms used in the data model into institution-specific terms having the same meaning.

4. The facility as claimed in claim 3, wherein the thesaurus module is adapted to identify at least one of work steps and sequences of work steps using input and output data associated with the work steps in a database.

5. The facility as claimed in claim 1, wherein the resource database contains, as actual resources in the institution, names of responsible persons and of equipment.

6. The facility as claimed in claim 1, wherein work steps performable in the institution are marked in the work step database.

7. The facility as claimed in claim 1, further comprising:
a checking module for checking the read in data model for resources which are not available in the institution and proposes at least one of alternative available resources and a process for outsourcing associated work steps.

8. The facility as claimed in claim 7, wherein the checking module includes a user interface with a suitable user environment for data input which an operator can use to input additional information for the outsourcing.

9. The facility as claimed in claim 7, wherein the checking module is designed such that it constantly or repeatedly checks the availability of resources in the institution and, in the event of resources becoming unavailable, automatically generates a report regarding portions of the implemented data model which are affected by the lack of availability.

10. The facility as claimed in claim 9, wherein, in the event of resources becoming unavailable, the checking module automatically prompts at least temporary alignment of the implemented data model by proposing one or more substitute processes.

11. The facility as claimed in claim 1, further comprising:
a scheduling module, to ensure prompt availability of resources listed in the data model.

12. The facility as claimed in claim 1, further comprising:
a plausibility module for comparing input and output data in the work steps and also the order of the work steps in the data model which has been read in and the data model which has been aligned, and generates a report in the event of discrepancies.

13. The facility as claimed in claim 1, further comprising:
an interaction module for, in the event of a work step not being found in the work step database or in the event of a title not being found in the thesaurus module, using a user environment to ask an operator whether the work step which has not been found needs to be transferred to the work step database or whether the title which has not been found needs to be transferred to the thesaurus module, and for transferring the work step which has not been found or the title which has not been found when instructed by the operator.

14. The facility as claimed in claim 1, wherein the provision module includes a graphical user interface for showing an operator the aligned data model in comparison with the data model which has been read in.

15. The facility as claimed in claim 14, wherein the provision module graphically highlights elements in a representation of the aligned data model which were at least one of able to be translated clearly, caused a lack of clarities or ambiguities and were not able to be mapped or translated.

16. The facility as claimed in claim 1, wherein the provision module includes an interface to a user interface in a workflow generation application, for at least one of correct by an operator and adding by the operator, to the aligned data model.

17. The facility as claimed in 1, wherein the facility is for adapting medical guidelines.

18. The facility as claimed in claim 2, further comprising a thesaurus module for translating terms used in the data model into institution-specific terms having the same meaning.

19. The facility as claimed in claim 18, further comprising a thesaurus module for translating terms used in the data model into institution-specific terms having the same meaning.

20. The facility as claimed in claim 8, wherein the checking module is designed such that it constantly or repeatedly checks the availability of resources in the institution and, in the event of resources becoming unavailable, automatically generates a report regarding portions of the implemented data model which are affected by the lack of availability.

21. The facility as claimed in claim 20, wherein, in the event of resources becoming unavailable, the checking module automatically prompts at least temporary alignment of the implemented data model by proposing one or more substitute processes.
